(19) United States
(12) Patent Application Publication
(10) Pub. No.: US 2010/0298630 A1
(43) Pub. Date: Nov. 25, 2010

(54) PELVIC FLOOR TREATMENTS AND RELATED TOOLS AND IMPLANTS

(76) Inventor: Shawn Michael Wignall, Minnetonka, MN (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 12/669,087

(22) PCT Filed: Dec. 5, 2008
(86) PCT No.: PCT/US08/13452

§ 371 Date: May 13, 2010

Related U.S. Application Data
(60) Provisional application No. 61/012,260, filed on Dec. 7, 2007.

(51) Int. Cl. A61F 2/02

(52) U.S. Cl. 600/37

(57) ABSTRACT

Described are implants, tools, and methods useful for treating pelvic conditions such as prolapse, incontinence, and others, by placing an implant to support pelvic tissue, the implants, tools, and methods involving placement of implants at locations within the pelvic region by placing an extension portion within tissue of the pelvic region.

### PRIORITY CLAIM

This application claims priority to U.S. Provisional Patent Application Ser. No. 61/012,260, filed on Dec. 7, 2007, titled PELVIC FLOOR TREATMENTS AND ASSOCIATED IMPLANTS, the entirety of which is incorporated herein by reference.

### FIELD OF THE INVENTION

The invention relates to apparatus and methods for treating pelvic conditions by use of a pelvic implant to support pelvic tissue. The pelvic conditions include conditions of the female or male anatomy, and specifically include treatments of female or male urinary and fecal incontinence; treatment of female vaginal prolapse conditions including enterocele, rectocele, cystocele, vault prolapse; conditions of the pelvic floor, and any of these conditions in combination. Particular examples of articles and tools described herein include: surgical implants that support pelvic tissue, and tools useful for placing the implants to support tissue of a pelvic region.

### BACKGROUND

Pelvic health for men and women is a medical area of increasing importance, at least in part due to an aging population. Examples of common pelvic ailments include incontinence (fecal and urinary), pelvic tissue prolapse (e.g., female vaginal prolapse), and conditions of the pelvic floor. Urinary incontinence can further be classified as including different types, such as stress urinary incontinence (SUI), urge urinary incontinence, mixed urinary incontinence, among others. Other pelvic tissue disorders include cystocele, rectocele, enterocele, and prolapse such as anal, uterine and vaginal vault prolapse. Pelvic disorders such as these can result from weakness or damage to normal pelvic support systems. Due to the lack of support, structures such as the uterus, rectum, bladder, urethra, small intestine, or vagina, may begin to fall out of their normal positions.

Conditions referred to as “conditions of the pelvic floor” include conditions caused by weakness or injury to pelvic floor muscles, including levator muscles. Specific conditions include levator avulsion in a female patient, and related conditions such as fecal or urinary incontinence, vaginal prolapse, conditions of the perineal body, conditions of the levator hiatus, levator ballooning, and combinations of two or more of these.

Pelvic conditions can be treated by various surgical and non-surgical methods. Non-surgical treatments for vaginal prolapse include pelvic muscle exercises, estrogen supplementation, and vaginal pessaries. The Perigee® system, developed by American Medical Systems located in Minnetonka, Minn., is a surgical product for the repair of anterior vaginal prolapse. The Apogee® system, developed by American Medical Systems is a surgical product for the repair of vaginal vault prolapse and posterior prolapse. Other commercially available products and medical devices are available for treating male urinary incontinence, female urinary incontinence, fecal incontinence, and conditions of the pelvic floor including but not limited to levator avulsion.

### SUMMARY

The present disclosure identifies pelvic implants, components of implants, related devices, systems and kits containing these, and methods of using these for treating pelvic conditions such as incontinence (various forms such as fecal incontinence, stress urinary incontinence, urge incontinence, mixed incontinence, etc.), vaginal prolapse (including various forms such as enterocele, cystocele, rectocele, apical or vault prolapse, uterine descent, etc.), conditions of the pelvic floor and result from weakness or trauma of pelvic floor muscles such as the levator (“levator ani”) or coccygeus muscle (collectively the pelvic floor), and other conditions caused by muscle and ligament weakness.

Exemplary methods treat male and female urinary incontinence. Other exemplary methods can involve treatment of vaginal prolapse, including anterior prolapse, posterior prolapse, or vault prolapse. The methods for female anatomy can be transvaginal, involving a single incision in the vaginal tissue, with no external incision. For male anatomy, methods of treating urinary incontinence can involve a single perineal incision.

Certain methods of treating female vaginal prolapse can provide for Level 1 support of the vaginal apex in combination with Level 2 support of medial vaginal sidewall tissue. In terms of vaginal prolapse, Level 1 vaginal tissue support relates to support of the top portion, or “apex” of the vagina. This section of tissue is naturally supported by the cardinal ligament that goes laterally to the ischial spine and crosses over medially to the sacrospinous ligament, and also by the uterosacral ligament that anchors into the sacrum. Level 2 support of vaginal tissue is support of tissue of the mid section of the vagina, below the bladder. This tissue is partially supported by the cardinal ligament but is predominantly supported by lateral fascial attachments to the arcus tendineus or white line. Level 3 support is that of the front end (sometimes referred to as the “distal” section) of the vagina right under the urethra. Natural support includes lateral fascial attachments that anchor into the obturator internus muscle.

Certain types of pelvic implants have in the past used bone anchors or soft tissue anchors. Various types of tissue fasteners are known, including, as examples, a self-fixating tip that is inserted into soft tissue and frictionally retained; soft tissue anchors; biologic adhesives; a soft tissue clamp that can generally include opposing, optionally biased, jaws that close to grab tissue; and opposing male and female connector elements that engage to secure an end of an extension portion to tissue. (See, e.g., International Patent Application No. PCT/US2007/014120, entitled “Surgical Implants, Tools, and Methods for Treating Pelvic Conditions,” filed Jun. 15, 2007, the entirety of which is incorporated herein by reference.)

The presently described implants and methods do not require bone or soft tissue anchors, but instead use novel, alternative method and structures for securing an implant to support tissue. Described are implants that include a dart at an end of an extension portion. The dart can engage a dart insertion tool in a manner to allow the dart insertion tool to place the dart through an incision in a patient then to a location near soft support tissue. The dart insertion tool can be used to push the dart to cause the dart to enter the soft support tissue, then pass the dart into or through the soft support tissue. A receiver tool includes a receiver that is capable of receiving the dart and engaging the dart for manipulation by the receiver tool.

Preferably, the dart can be pushed into soft support tissue at a first surface (at an insertion location) and can then be manipulated to pass into and through a tissue path produced by further pushing the dart using the dart insertion tool. The dart passes within the tissue (e.g., tunnels through a length of the tissue), then exits the tissue at a location near the insertion location and on the same side of soft support tissue. The result is that the extension portion becomes looped or tunneled through the tissue, entering at the insertion location, passing laterally through a length of the soft support tissue mass, then exiting at an exit location that is on the same side of the soft support tissue as the insertion location.

The receiver may function in any useful manner. In one embodiment, a receiver penetrates into the soft support tissue at the exit location, creates a portion of a tissue path within the soft support tissue, and engages the dart at a location internal to the soft support tissue. The dart insertion tool can then be withdrawn away from the dart and the dart can be manipulated by the receiver tool, e.g., by the receiver pulling the dart out of the exit location. The length of the extension portion between the attached tissue support portion and the insertion location can then be adjusted by changing the amount of extension portion that extends through the soft support tissue.

In another embodiment, the receiver contacts the soft support tissue at the exit location without penetrating into the soft support tissue. The dart insertion tool passes the dart through the soft support tissue and to the receiver, located at the surface of the soft support tissue at the exit location. The dart becomes engaged with the receiver at a location external to the soft support tissue. The dart insertion tool can then be withdrawn away from the dart and the dart can be manipulated by the receiver tool, e.g., by the receiver pulling away from the exit location. The length of the extension portion between the attached tissue support portion, and the insertion location, can then be adjusted by changing the amount of extension portion that extends through the soft support tissue.

The dart insertion tool and the receiver tool may be separate tools, or may be different elements or portions of a single tool. If the latter, the dart insertion tool may be a jaw of a forceps or pliers and the receiver tool may be an opposing jaw of the same tool.

An aspect of the invention relates to a pelvic implant system. The system includes a pelvic implant, a dart insertion tool, and a receiver. The implant includes a tissue support portion, an extension portion, and a dart connected to the extension portion. The dart is capable of engaging the dart insertion tool, and the dart is capable of engaging the receiver.

In another aspect the invention relates to a pelvic implant system that includes a pelvic implant, a dart insertion tool, and a receiver. The pelvic implant includes a tissue support portion, an extension portion, and a dart connected to the extension portion. The dart includes insertion tool engaging means capable of engaging a distal end of the dart insertion tool, and receiver engaging means capable of engaging the receiver.

In another aspect the invention relates to a method for treating a pelvic condition. The method includes: providing an implant that includes a tissue support portion, an extension portion, and a dart attached to the extension portion; placing the tissue support portion in contact with pelvic tissue, extending the extension portion to soft support tissue, inserting the dart into the soft support tissue at a surface of the soft support tissue, passing the dart through soft support tissue, and causing the dart to exit the soft support tissue from a surface on the same side of the soft support tissue into which the dart was inserted.

In yet another aspect, the invention relates to a method for treating a pelvic condition. The method includes: providing an implant that includes a tissue support portion and an extension portion; placing the tissue support portion in contact with pelvic tissue, extending the extension portion to soft support tissue, inserting the extension portion into the soft support tissue at an insertion location, passing the extension portion through the soft support tissue, and causing the extension portion to exit the soft support tissue at an exit location on the same side of the soft support tissue as the insertion location. The distance between the insertion location and the exit location is not greater than 2.5 centimeters.

Still other aspects of the invention relate to methods of manipulating a pelvic implant system. Such a method can include providing a pelvic implant system as described herein, including a dart, a dart insertion tool and a receiver; engaging the dart and the dart insertion tool; and engaging the dart and the receiver.

The following patent documents are incorporated herein by reference: US Patent Publication No. US 2004/0039453 A1; US Patent Publication No. US 2005/0250977 A1; US Patent Publication No. US 2005/0245787 A1; U.S. Pat. No., 6,652,450; U.S. Pat. No. 6,612,977; U.S. Pat. No. 6,802,807; U.S. Pat. No. 7,048,682; U.S. Pat. No. 6,641,525; U.S. Pat. No. 6,911,003; U.S. Pat. No. 7,070,556; U.S. Pat. No. 6,354,991; U.S. Pat. No. 6,896,651; U.S. Pat. No. 6,652,449; U.S. Pat. No. 6,862,480; U.S. Pat. No. 6,712,772; and PCT Application WO 2007/149348, filed Jun. 15, 2007, titled “Surgical Implants, Tools and Methods for Treating Pelvic Conditions” (Attorney Docket No. AMS-3419-PCT).

### BRIEF DESCRIPTION OF DRAWINGS

FIG. 1 shows an embodiment of a dart according to the description.

FIGS. 2A, 2B, and 2C show embodiments of darts and dart insertion tools according to the description.

FIGS. 3A and 3B show an embodiment of a receiver according to the description.

FIGS. 4A, 4B, and 4C show embodiments of darts and dart insertion tools according to the description, used in method steps of the description.

FIGS. 5A, 5B, and 5C show an embodiment of a receiver according to the description.

FIGS. 6A, 6B, and 6C show embodiments of receivers according to the description.

FIG. 7 shows an embodiment of a dart insertion tool and a receiver tool, as described.

FIG. 8 shows an embodiment of a dart insertion tool and a receiver tool, as described.

FIGS. 9A through 9F show steps of an embodiment of a described method.

FIGS. 10A through 10F show steps of an embodiment of a described method.

FIG. 11 illustrates an embodiment of an extension portion comprising a clip to secure an adjusted position of an extension portion.

FIGS. 12A, 12B, 12C, and 12D illustrate exemplary embodiments of implants.

All drawings are schematic and not to scale.

### DETAILED DESCRIPTION

The following description is meant to be illustrative only and not limiting. Other embodiments of this invention will be apparent to those of ordinary skill in the art in view of this description.

The invention involves surgical instruments, assemblies, and implantable articles for treating pelvic disorders such as fecal or urinary incontinence, including stress urinary incontinence (SUI), prolapse, conditions of the pelvic floor, and the like. An implant can be implanted in a male or a female patient to treat a condition such as urge incontinence; stress urinary incontinence; mixed incontinence; overflow incontinence; functional incontinence; fecal incontinence; prolapse (e.g. vaginal or uterine); enterocele (e.g. of the uterus); rectocele; cystocele; anatomic hypermobility; conditions of the pelvic floor caused by weakness or trauma of pelvic floor muscles such as the levator (“levator ani”) or coccygeus muscle (collectively the pelvic floor); other conditions caused by muscle and ligament weakness; and combinations of these.

An implant includes a tissue support portion that can be used to support pelvic tissue such as the urethra (which includes the bladder neck), bladder, rectum, vaginal tissue (Level 1, Level 2, Level 3, or combinations of these), pelvic floor tissue, etc. During use, the tissue support portion is typically placed in contact with and attached to tissue to be supported (“pelvic tissue”), such as by attachment using one or more sutures. An implant additionally includes one or more extension portion attached to the tissue support portion, and optionally one or more scaffold portion (a type of “extension portion”) attached to a tissue support portion or an extension portion.

An example of a particular type of pelvic implant is the type that includes supportive portions including or consisting of a tissue support portion and either two, four, or six elongate extension portions extending from the tissue support portion. An implant that has exactly two extension portions can be of the type useful for treating, e.g., urinary incontinence, anterior vaginal prolapse, fecal incontinence, posterior vaginal prolapse; an implant having four or six extension portions can be useful for treating any of these or a combinations of these conditions. The term “supportive portions” refers to portions of an implant that function to support tissue after the implant has been implanted, and specifically includes extension portions and tissue support portions, and does not include optional or appurtenant features of an implant such as a sheath, tensioning member (e.g., suture), or dart.

The implants and methods include a dart attached to an extension portion. A “dart,” as the term is used herein, refers to any structure, including structures commonly referred to as a “dart,” or otherwise, that includes structure or functionality of a “dart” as that term is used herein.

A dart can attach to an extension portion, e.g., a distal end of an extension portion can attach to a proximal dart portion. A dart can attach to an extension portion by any known or useful attachment mechanism. Examples include adhesive, molding (e.g., “overmolding”) or thermoforming a dart onto an extension portion, use of a mechanical fastener such as a screw or staple, or any other method of securing an extension portion (e.g., an end of an extension portion) to a dart. Preferred darts can be of a polymeric (e.g., plastic, thermoplastic, thermoforming, or thermosetting) material and can be molded directly onto an extension portion, preferably at the distal end of the extension portion. Attaching the dart at the “end” or “distal end” refers to attachment of the dart at a general location on the extension portion that is spaced distally from the tissue support portion; the attachment need not be at the exact “end” or tip of the extension portion.

A dart can include a proximal dart portion and a distal dart portion. A proximal dart portion is a dart portion generally on a side of the dart nearest the tissue support portion, or nearest an attachment of the dart to the extension portion, or both; a distal dart portion is a dart portion generally distal to the proximal dart portion. A proximal dart portion can include a surface that engages a distal end of an insertion tool. For example, a proximal dart portion may include a proximal surface facing the extension portion, with which a surface of a dart insertion tool distal end may be engaged during use. The engagement allows the insertion tool to push the dart in a direction to produce a tissue path in soft support tissue, generally a direction of the distal dart portion leading through the soft support tissue. Alternately, or in addition, for improved engagement between a dart and an insertion tool distal end, a dart may include a channel or opening (preferably but not necessarily along a longitudinal axis of the dart) that may receive a correspondingly sized and shaped elongate structure of a dart insertion tool distal end. Still differently, a dart may include an exterior surface that engages a dart insertion tool distal end. Regardless of the specific structure of the engagement, the engagement may be loose or mechanically, (e.g., securely, releasably) actuated. A loose engagement may be based merely on friction, e.g., allowing the dart to easily slide on and off of the dart insertion tool distal end. A mechanically actuated engagement may include a detent or other securing mechanism by which the dart insertion tool distal end may selectively engage and disengage the dart, e.g., by use of an actuator at a proximal end of the dart insertion tool, such as at a handle.

An exemplary distal dart portion can include a surface that facilitates passage of the dart through soft support tissue when the dart is pushed into or through the soft support tissue. The distal dart portion can include, e.g., a sharpened (e.g., pointed) surface capable of cutting, breaking, severing, or splitting, etc., soft support tissue, or otherwise capable of initiating or propagating a tissue path in soft support tissue. The distal dart portion can alternately or additional include a deflecting surface that deflects soft tissue sidewise or otherwise away from a longitudinal axis of the dart to produce or enlarge a tissue path to allow passage of the dart through the soft support tissue. A deflecting surface may generally be a surface that initiates at a sharpened surface of the distal dart portion, e.g., a point or a line, and that expands or enlarges while extending in a direction toward the proximal dart portion. A deflecting surface may be tapered, slanted, curved, angled, or rounded, etc.

The dart also includes a surface that engages a receiver. This surface generally can include an extension, recession, a curved, jagged, or sharp or angled surface, or another type of protruding or recessed surface or edge that can contact and engage a corresponding (e.g., opposite) surface of the receiver to preferably produce a secure engagement between the dart and the receiver. The dart surface that engages a receiver may be a shoulder, ledge, protuberance, bulge, protrusion, detent, shelf, ridge, bump, depression, jagged extension, or the like, located at one or more internal surface of the dart or at one or more external surface of the dart, e.g., at one or multiple or continuous locations around an inner or outer surface or circumference of a dart surface. The dart surface that engages a receiver may be fixed, may be biased and may move by deflection during engagement with the receiver, or may be associated with a mechanical actuator (e.g., at a handle of the tool) to selectively engage and disengage upon use of the actuator.

A “secure engagement” between a receiver and a dart is an engagement that allows the dart to be manipulated by the receiver by use of a receiver tool, according to methods described herein. A secure engagement may allow the receiver and receiver tool to be used to pull either or both of the dart and an extension portion attached to the dart through soft support tissue to allow placement and adjustment of the extension portion within the soft support tissue. This in turn allows for placement and adjustment of a tissue support portion attached to the extension portion, as described. Examples of secure engagements include locking engagements, snap-fit engagements, and the like, that allow the receiver to manipulate the dart. Optionally, a secure (e.g., snap-fit or locking) engagement may be permanent or semi-permanent, such as an engagement that is sufficiently secure to prevent manual separation of the dart and receiver.

A receiver can be an integral part of a receiver tool or can be a separate piece that removably engages a receiver tool.

The receiver includes a surface that engages a dart. This surface generally can include an extension, recession, a curved, jagged, or sharp or angled surface, or another type of protruding or recessed surface or edge that can contact and mate with a corresponding (e.g., opposite) recessed or extended surface of the receiver to produce a secure engagement between the dart and the receiver. The receiver surface that engages a dart may be a shoulder, ledge, protuberance, bulge, protrusion, detent, shelf, ridge, bump, depression, jagged extension, or the like, located at one or more internal surface of the receiver or at one or more external surface of the receiver, e.g., at one or multiple or continuous locations around an inner or outer circumference of a receiver surface. The receiver surface that engages a dart may be fixed, may be biased and may move by deflection during engagement with the receiver, or may be associated with a mechanical actuator (e.g., at a handle of the tool) to selectively engage and disengage upon use of the actuator.

Optionally, a receiver can include a distal receiver portion that is capable of engaging or penetrating soft support tissue. In certain embodiments of the described methods, a receiver penetrates soft tissue. For these exemplary methods a distal receiver portion can include structure that can form a tissue path. Such a distal receiver portion can include, e.g., a sharpened or pointed surface capable of cutting, breaking, severing, or splitting, soft support tissue, or otherwise capable of initiating or propagating a tissue path in soft support tissue. The distal receiver portion can alternately or additional include a deflecting surface that deflects soft tissue sidewise or otherwise away from a longitudinal axis of the receiver to produce or enlarge a tissue path to allow passage of the receiver through soft support tissue. A deflecting surface may generally be a surface that initiates at a sharpened surface at the distal receiver portion, e.g., a point or a line, and that expands or enlarges while extending in a direction toward the proximal receiver portion. A deflecting surface may be tapered, slanted, curved, angled, or rounded, etc.

In other embodiments of described methods a receiver engages but does not penetrate soft tissue. For these exemplary methods, a distal receiver portion can include a frictional surface that frictionally engages soft support tissue in a manner that allows the receiver to engage a surface of the soft support tissue at an exit location and orient the tissue to allow a dart to pass through the soft support tissue and upon exiting the soft support tissue, to meet the receiver for engagement, external to the soft support tissue. Once the dart is securely engaged by the receiver, the receiver can manipulate the dart and the attached extension portion. For example, a distal receiver portion can include a frictional surface that includes teeth, ridges, or other sharpened or pointed surfaces (e.g., pyramidal surfaces, elongate ridges) that frictionally engage a surface of soft support tissue to allow the soft support tissue to be held, pinched, or grasped (e.g., between two opposing jaws of a forceps or other tools as described herein).

A dart and a receiver can be made out of any useful material, generally including materials that can be molded or formed to a desired structure and connected to or attached to an extension portion. Useful materials can include plastics such as polyethylene, polypropylene, and other thermoplastic or thermoformable materials, as well as metals, ceramics, and other types of biocompatible materials. Exemplary materials for a dart include thermoformable or thermosetting plastics or other polymeric materials, such as polyethylene, polypropylene, and the like. Polymeric materials can be preferred based on ease of preparing a molded, e.g., overmolded, receiver at an end of an extension portion. Exemplary materials for a receiver include these same thermoformable or thermosetting plastics or other polymeric materials, also preferred based on ease of preparing a molded or overmolded receiver onto an end of an extension portion; other exemplary receivers may be formed from a metal, such as if the receiver is integral to a receiver tool.

A tissue support portion is designed to support a specific type of pelvic tissue such as the urethra, bladder, or vaginal tissue (anterior, posterior, apical, etc.), rectum, tissue of the pelvic floor such as levator muscle, etc. The tissue support portion can be sized and shaped to contact the desired tissue when installed, e.g., as a “sling” or “hammock,” to contact and support pelvic tissue.

Extension portions are pieces of material, generally elongate or otherwise extended from a tissue support portion, and that are useful to pass through soft support tissue to thereby provide support for the tissue support portion and the supported pelvic tissue. One or multiple (e.g., one, two, four, or six) extension portions can extend from a tissue support portion for attachment to soft support tissue.

An implant can optionally include a scaffold portion, (which is a type of extension portion) that can be extended internally within a patient and secured to tissue of a pelvic region or to a location of the implant, and used to support a tissue support portion or another extension portion. A scaffold portion can have two ends. Either end can include a dart, as described, to attach the scaffold portion internally to tissue of the pelvic region.

Exemplary implants can be made of materials and may be generally shaped and sized with certain individual features that may be found in previous implants, but can be modified to include a dart as described herein and adapted for use according to methods described herein. The length of the extension portion may be modified (e.g., increased) from previous implants, to allow for the extension portion to pass through soft support tissue and be manipulated for adjustment. An implant can have features described in the following exemplary documents: U.S. patent application Ser. No. 10/834,943, filed Apr. 30, 2004 (Publ. No. 2005-0245787); U.S. patent application Ser. No. 10/306,179, filed Nov. 27, 2002; U.S. patent application Ser. No. 11/347,063, filed Feb. 3, 2006 (Publ. No. 2006-0195011); U.S. patent application Ser. No. 11/347,596, filed Feb. 3, 2006 (Pub. No. 2006-0195010); U.S. patent application Ser. No. 11/347,553, filed Feb. 3, 2006 (Pub. No. 2006-0235262); U.S. patent application Ser. No. 11/347,047, filed Feb. 3, 2006 (Pub. No. 2006-0287571); U.S. patent application Ser. No. 11/346,750, filed Feb. 3, 2006 (Pub. No. 2006-0195007); U.S. patent application Ser. No. 11/398,368, filed Apr. 5, 2005 (Pub. No. 2006-0260618); U.S. patent application Ser. No. 11/243,802, filed Oct. 5, 2005 (Pub. No. 2006-0122457); U.S. patent application Ser. No. 10/840,646, filed May 7, 2004 (Pub. No. 2005-0250977); and International patent application number PCT/US2006/028828, having an International Filing Date of Jul. 25, 2006; International Application No. PCT/US2007/004015 entitled “SURGICAL ARTICLES AND METHODS FOR TREATING PELVIC CONDITIONS,” filed Feb. 16, 2007; International Application No. PCT/US2007/016760 entitled “SURGICAL ARTICLES AND METHODS FOR TREATING PELVIC CONDITIONS,” filed Jul. 25, 2007; and International Application No. PCT/US2008/000033 entitled “METHODS FOR INSTALLING SLING TO TREAT FECAL INCONTINENCE, AND RELATED DEVICES,” filed Jan. 3, 2008, International Application No. PCT/US2008/09066 entitled “PELVIC FLOOR TREATMENTS AND RELATED TOOLS AND IMPLANTS,” filed Jul. 25, 2008; and WO 2008/124056 A1, entitled “KIT FOR LEVATOR AVULSION REPAIR,” filed Apr. 4, 2008, the entireties of each of these disclosures being incorporated herein by reference.

Examples of commercial implants include those sold by American Medical Systems, Inc., of Minnetonka Minn., under the trade names Apogee®, Perigee®, and Elevate™ for use in treating pelvic prolapse (including vaginal vault prolapse, cystocele, enterocele, etc.), and Sparc®, Bioarc®, Monarc®, MiniArc™, and AdVance® for treating urinary incontinence. Implants useful according to the present description can include one or more features of these commercial implants, including generally similar tissue support portions and extension portions, but modified to include a dart as described.

An implant may include portions, pieces, or segments, that are synthetic or of biological material (e.g., porcine, cadaveric, etc.). Extension portions may be, e.g., a synthetic mesh such as a polypropylene mesh. A tissue support portion may be synthetic (e.g., a polypropylene mesh) or biologic.

Examples of implants for treating vaginal prolapse (e.g., anterior vaginal prolapse, posterior vaginal prolapse, vaginal vault prolapse) can include a central support portion and from two to four to six extension portions, and may take the form of an integral piece of mesh or other implant material, or multiple pieces of mesh or other implant material attached in a modular fashion. See, e.g., Assignee's copending U.S. patent application Ser. Nos. 11/398,369; 10/834,943; 11/243,802; 10/840,646; PCT/2006/028828; among others. Particularly useful examples of implants for treating vaginal prolapse using any one or a combination of devices or methods as described herein can be implants described in Assignee's copending International Patent Application No. PCT/US2007/014120, entitled “SURGICAL IMPLANTS, TOOLS, AND METHODS FOR TREATING PELVIC CONDITIONS,” filed Jun. 15, 2007, the entirety of which is incorporated herein by reference.

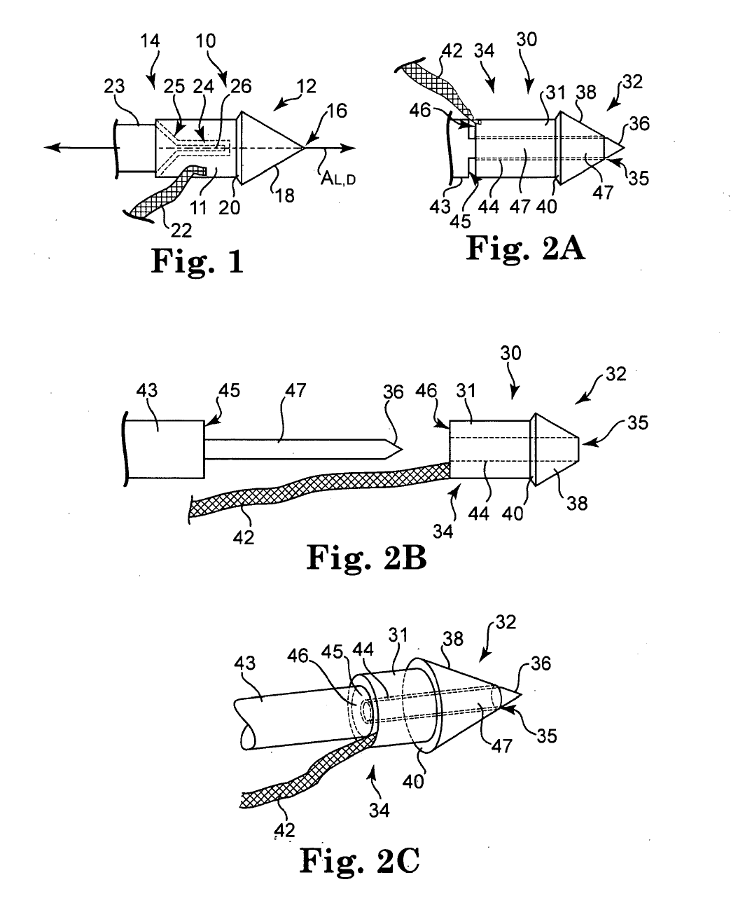

One embodiment of a dart attached to a distal end of a mesh extension portion is illustrated at FIG. 1. Referring to FIG. 1, a side view of dart 10 is shown. This type of dart in general can be a structure connected to an extension portion that can be inserted into and passed through soft support tissue (e.g., muscle tissue, tendon tissue, or ligament tissue) in a manner that will cause the extension portion to be passed through, e.g., looped or threaded, through the soft support tissue. A proximal end 14 includes cylindrical base 11, which contains internal structure (internal channel 24 and slanted proximal surface 25) that engages corresponding structure of a tip of a distal end 23 of a dart insertion tool. A distal end of extension portion 22 attaches at an exterior surface of base 11. Dart 10, engaged at the distal end of dart insertion tool 23, can be pushed by dart insertion tool 23 into and through soft support tissue. Sharpened surface (pointed dart tip) 16 can puncture a surface of soft support tissue and thereafter initiate a tissue path. Proximal to pointed dart tip 16 is deflecting surface 18 in the form of a round, tapered, three-dimensional, arrowhead-shaped or cone-shaped surface. At the proximal end of deflecting surface 18 is extension 20 (e.g., “ledge” or “shoulder”), which connects a trailing boundary of deflecting surface 18, to base 11. Extension 20 can be used to produce a secure engagement between dart 10 and a receiver. The line designated A_{L,D} refers to the longitudinal axis of the dart.

FIG. 2A illustrates a variation of this type of dart. Referring to FIG. 2A, a side view of dart 30 is shown. A distal end of extension portion 42 attaches to base 31 of dart 30. Proximal dart end 34 includes cylindrical base 31 and internal channel 44 that engages corresponding elongate tip 47 of distal end 43 of a dart insertion tool. Channel 44 extends through the entire length of dart 30, ending at aperture 35 at the distal tip of distal dart portion 32. Distal shoulder surface 45 of the dart insertion tool engages proximal surface or “shoulder” 46 of base 31, the engagement allowing the dart insertion tool to push dart 30 for placement and for use in methods as described. Elongate tip 47 of the dart insertion tool extends into channel 44 to additionally engage dart 30; pointed tip 36 extends beyond distal dart portion 32, through aperture 35, and functions to initiate a tissue path. Distal dart portion 32 includes deflecting surface 38 in the form of a tapered, three-dimensional, arrowhead-shaped or cone-shaped surface. At the proximal end of deflecting surface 38 is extension 40 (e.g., “ledge” or “shoulder”), which connects a trailing boundary of surface 38 to base 31. Extension 40 can be used to produce a secure engagement between dart 30 and a receiver.

FIG. 2B is an alternate view of the dart and dart insertion tool of FIG. 2A. FIG. 2B illustrates a side view of distal end of dart insertion tool 43 and elongate tip 47, not engaged with dart 30.

FIG. 2C is an alternate view of the dart and insertion tool of FIG. 2A. FIG. 2C illustrates a perspective view of distal end of dart insertion tool 43 and elongate tip 47, engaged with dart 30.

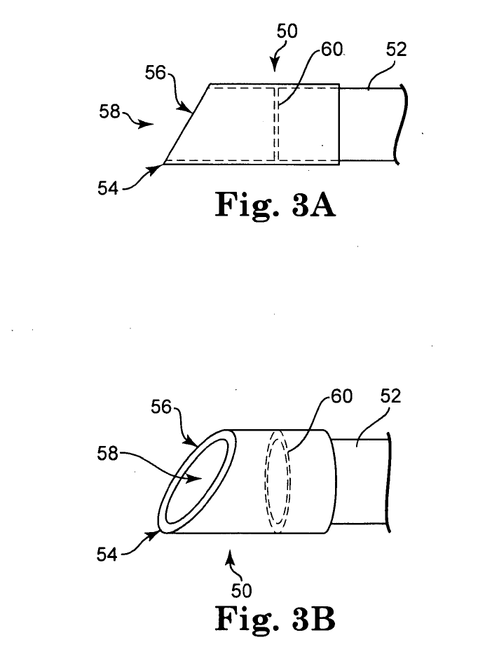

FIG. 3A is a side-view illustration of an embodiment of a receiver. Receiver 50 is attached, e.g., integrally and permanently, to distal end 52 of a receiver tool. Receiver 50 is generally the shape of a hollow cylinder having a slanted distal (deflecting) surface 54, surrounding aperture 58. Leading tip 54 is a pointed or sharpened surface that can initiate a tissue path (when receiver 50 is pushed using the receiver tool), and slanted surface 56 surrounding aperture 58 deflects tissue to open or propagate the tissue path to allow passage of the cylindrical receiver. Aperture 58 is sized to accept a dart. Ridge 60 can be a bump, ledge, shoulder, extension, recession, etc., or other surface capable of engaging a corresponding surface of a dart (e.g., surface 20, 40 of darts 10, 30, respectively) to produce an engagement, preferably a secure engagement, between the dart and receiver 50. FIG. 3B shows a side perspective view of receiver 50.

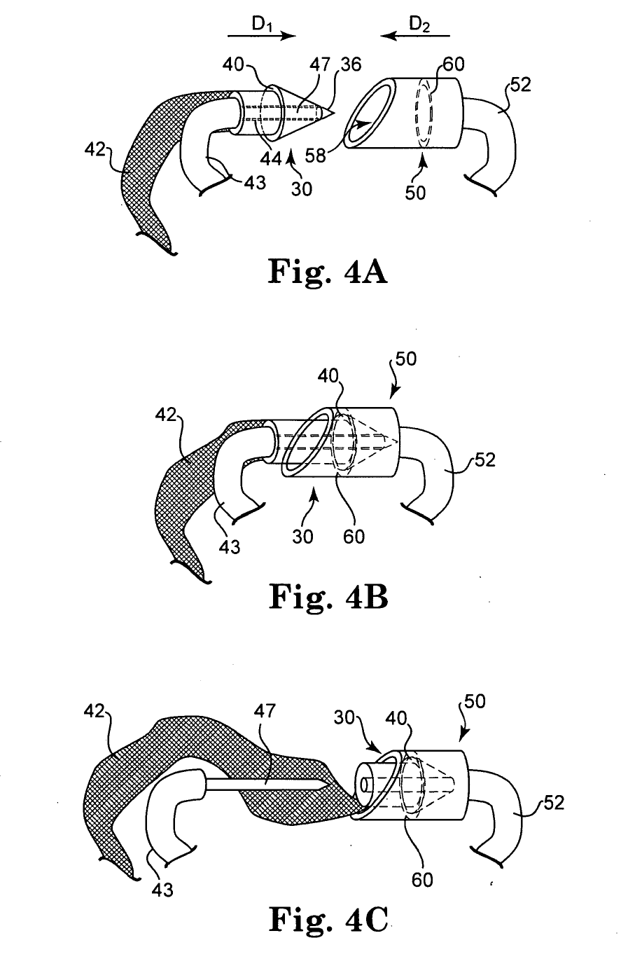

FIGS. 4A, 4B, and 4C illustrate the action of transferring a dart as illustrated at FIGS. 2A and 2B, from a dart insertion tool to a receiver as illustrated at FIGS. 3A and 3B. (While these specific dart, receiver, and tool structures are illustrated, the steps apply to other dart, receiver, and tool structures described or illustrated herein.) FIG. 4A shows dart 30 engaged with a distal end of dart insertion tool 43, with elongate tip 47 being located within channel 44 and pointed tip 36 extending from a distal dart portion. Dart 30 is attached to a distal end of extension portion 42. Dart 30 approaches receiver 50 along a longitudinal axis shared by dart 30 and receiver 50; dart 30 moves in direction D1 and receiver 50 optionally moves in direction D2. Dart 30 is aligned to enter aperture 58.

FIG. 4B illustrates dart 30 securely engaged with receiver 50. Dart 30 has been advanced into aperture 58 a distance to cause surface (“extension” or “ledge”) 40 to engage ridge 60. The engagement between these surfaces (40 and 60) produces a snap-fit engagement that is a secure engagement. FIG. 4C illustrates dart 30 securely engaged with receiver 50. Distal end of dart insertion tool 43 and elongate tip 47 of the dart insertion tool are withdrawn from dart 30. Receiver 50, and attached receiver tool 52, can be used to manipulate dart 30, to thereby manipulate extension portion 42.

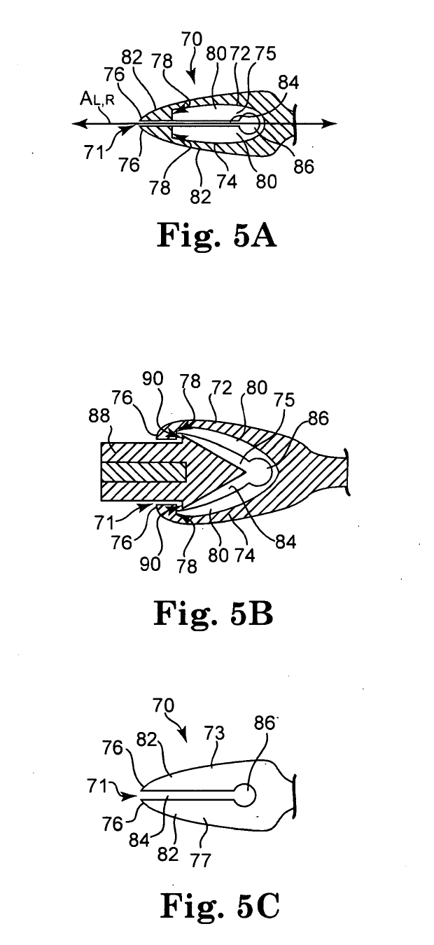

FIG. 5A is a side, cut-away view of another embodiment of a receiver. Receiver 70 is of a general type that includes deflectable, opposed lobes (72, 74) (or “jaws”) that deflect (outward as illustrated, but alternately inward). The lobes are biased in the position shown at FIGS. 5A (and 5C). Receiver 70 includes four lobes; lobes 72 and 74 as illustrates in cross section at FIGS. 5A and 5B are the “far” two lobes of receiver 70 as depicted; two “near” lobes 73 and 77 are illustrated at side view 4C of receiver 70. The lobes are separated along adjacent lengths by length-wise slots 84, and at their distal ends define aperture 71. Expansion 86 at the proximal end of slot 84 is an optional structure useful if necessary to facilitate deflection of lobes 72 and 74. The lobes are capable of deflecting upon contact with a sharpened or deflecting surface of a dart (or insertion tool) moving toward and into receiver 70 along longitudinal axis A_{L,R}. As a sharpened or deflecting surface of a dart advances through aperture 71 along the longitudinal axis and in a direction toward receiver 70, lobes 72 and 74 deflect away from the longitudinal axis, A_{L,R}, causing aperture 71 and cavity 75 to expand. As the dart advances farther into cavity 75, lobes 72, 74 may return, partially (e.g., snap) back in the direction of their biased orientation, tightening around the dart to produce a secure engagement between the dart and receiver 70. A receiver such as receiver 70 may include any number of lobes. Each lobe includes a sharpened distal end 76 that can be sufficiently pointed (singly, or collectively) or sharpened to penetrate tissue as receiver 70 is pushed from the opposite (proximal) end. Each lobe also includes deflecting surface 82, shown to be tapered and rounded, to propagate a tissue path. Internal cavity 75 is defined by internal surfaces (80) of the lobes; also within cavity 75, a distal portion of each lobe includes an internal surface 78 (facing in a proximal direction relative to the receiver) for engaging a surface of a dart to produce a secure engagement.

FIG. 5B illustrates receiver 70 in secure engagement with dart 88 (which may be, e.g., a dart of the type shown at FIG. 1A or 2A). Lobes 72 and 74 of receiver 70 have been deflected to enlarge aperture 71 and cavity 75 to accommodate dart 88. This also causes slot 84 to open to produce a larger space between the deflected lobes. A distal dart portion of dart 88 has entered cavity 75. A base of dart 88 is held by the lobes within expanded aperture 71. Receiver 70, at each lobe, includes multiple surfaces (“shoulders” or “ledges” 78) that engage opposing surfaces (90) of the dart to produce a secure engagement. More specifically, each of surfaces 78 engages a surface (opposing “shoulder” or “ledge” 90) of dart 88, to produce a permanent or semi-permanent snap-fit, secure engagement between receiver 70 and dart 88.

FIG. 5C is a side view of receiver 70.

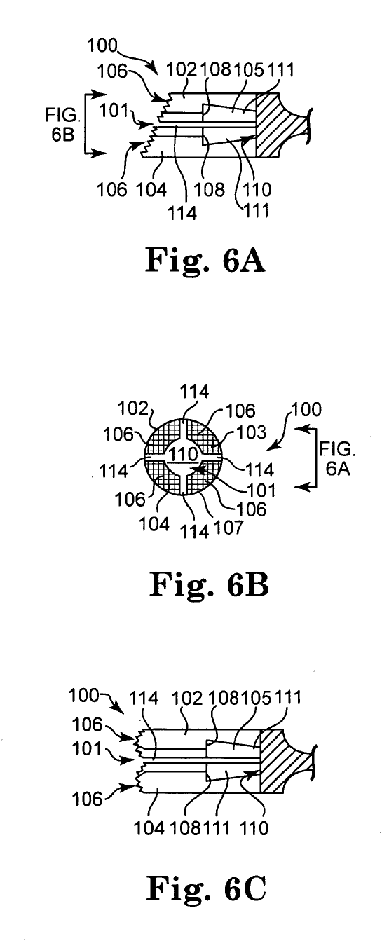

FIG. 6A is a side, cut-away view of another embodiment of a receiver; FIG. 6B is an end view of the receiver. Receiver 100 is of a general type that includes deflectable, opposed lobes (102, 104) (or “jaws”) that deflect (outward as illustrated, but alternately inward). The lobes are biased in the position shown at FIG. 6A. Receiver 100 includes four lobes; lobes 102 and 104 are illustrates in cross section at FIG. 6A are the “far” two lobes of receiver 100 as depicted; two “near” lobes 103 and 107 are illustrated at end view 6B of receiver 100. The lobes are separated along adjacent lengths by length-wise slots 114, and at their distal ends define aperture 101. Each lobe includes a distal surface 106 that is capable of frictionally contacting soft tissue. Surfaces 106 may include a rough, non-smooth surface such as a surface that includes sharpened or pointed ridges, pyramids, teeth, etc., that allows non-penetrating engagement with smooth soft tissue. As illustrated, the distal surface (surfaces 106, in aggregate) of receiver 100 is flat but angled (i.e., slanted and not perpendicular) relative to the longitudinal axis of receiver 100. Alternate embodiments can include distal surfaces 106 that are rounded (see FIG. 6C), flat, or perpendicular to the longitudinal axis of receiver 100, or otherwise shaped to facilitate frictional engagement with soft tissue.

In use, surfaces 106 contact a surface of soft support tissue at one end of a tissue path and can hold the soft support tissue in place while an opposed insertion tool (e.g., a jaw of a forceps) directs a dart into and through the soft tissue; as the dart, pushed by the dart insertion tool, exits the tissue, the dart meets receiver 100.

Still referring to FIGS. 6A and 6B, the lobes of receiver 100 are capable of deflecting upon contact with a sharpened or deflecting surface of a dart (or insertion tool) (not shown) along a longitudinal axis of receiver 100. As the leading or deflecting surface of a dart advances through aperture 101 along the longitudinal axis and in a direction toward receiver 100, lobes 102, 103, 104, and 107 deflect away from the longitudinal axis causing aperture 101 and cavity 105 to expand. As the dart advances farther into cavity 105, lobes 102, 103, 104, and 107 may return, partially (e.g., snap) back toward their biased orientation, tightening around the dart to produce a secure engagement between the dart and receiver 100.

A receiver such as receiver 100 may include any number of lobes. Each lobe includes a distal end surface 106 that can be surfaced to engage soft tissue as receiver 100 is manipulated (e.g., pushed) from the opposite (proximal) end using a receiver tool. Internal cavity 105 is defined by internal lobe surfaces 111, and proximal surface 110; also within cavity 105, a distal portion of each lobe includes an internal surface 108 (a “ledge,” “shoulder,” or “extension,” facing in a proximal direction relative to the receiver) for engaging a surface of a dart to produce a secure engagement.

FIG. 6C shows receiver 100 as in FIGS. 6A and 6B, modified to include rounded surfaces 106 at distal ends of each of lobes 102, 103, 104, and 107. Other than that difference the structure, function, and operability of receiver 100 are as described.

The implants can be implanted into a patient by use of various different types of surgical tools, including tools generally referred to as “dart insertion tools” useful to engage, manipulate, and place a dart, and “receiver tools” useful to engage, manipulate, and place a receiver. A receiver tool can be separate from a dart insertion tool, or a dart insertion tool can be combined into a single tool with the receiver tool. Various types of tools for manipulating implants, connectors, soft tissue anchors, etc., are known, and these general types of tools, modified according to the present description, can be used according to the present description to manipulate a dart or a receiver for delivery of an implant into a pelvic region of a patient.

One example of a dart insertion tool useful to engage a dart can include a handle, an elongate shaft or “needle,” and a needle distal end that includes a surface that engages a dart, preferably at a proximal dart portion. An example of a receiver tool can be in the form of a similar tool including a handle, an elongate shaft or “needle,” and a needle distal end that engages a receiver or includes an integral receiver permanently or integrally constructed at the distal end of the shaft. Alternate tools can be in the form of a “forceps” or “pliers” that includes two opposing jaws: one jaw can be considered an insertion tool to engage a dart and the opposing jaw can be the receiver tool useful to engage or be integral with the receiver.

Examples of useful needle-type tools for manipulating a dart or a receiver include those types of tool that generally include a thin elongate shaft (e.g., needle); a handle attached to one end (a proximal end) of the shaft; and a distal end of the shaft adapted to engage a dart or a receiver (or be integral with the receiver). According to exemplary methods of the invention, two needle-type tools can be used together, a dart insertion tool to manipulate the dart and a receiver tool to manipulate a receiver. A dart insertion tool can manipulate the dart through a vaginal, a medial (e.g., perineal), abdominal, laparoscopic, or other incision to reach soft support tissue; a receiver tool can be inserted through the same incision to locate the receiver in the same region of soft support tissue, and on the same “side” of the soft support tissue. The dart insertion tool can place the dart within or through soft support tissue; the receiver tool may optionally penetrate the soft support tissue; and the dart can be transferred from the dart insertion tool to the receiver. Both tools can be designed, shaped, and sized, to include an elongate shaft that may be straight or that may be curved in two or three dimensions, that can be inserted through a vaginal incision (for female anatomy) or through a perineal incision (for male anatomy), and extend from that incision to or through pelvic tissue for manipulation and placement of a dart and extension portion of an implant, as described.

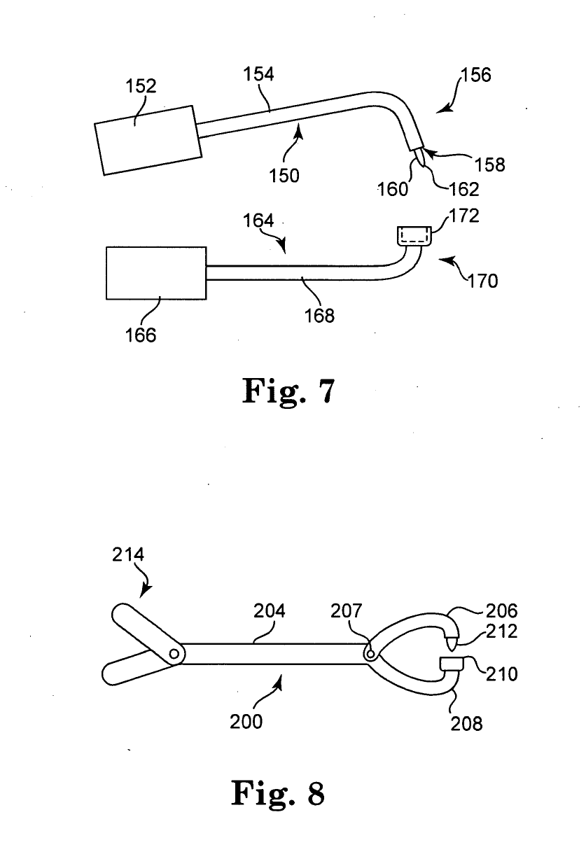

FIG. 7 illustrates one embodiment of a set of tools. Dart insertion tool 150 includes handle 152, shaft or “needle” 154, and distal end 156. Shoulder 158 can abut a proximal dart surface and elongate tip or extension (e.g., in the form of a “pin”) 160, with optional pointed or sharpened end 162, can fit within or through a channel of a dart. Receiver tool 164 includes handle 166, shaft or “needle” 168, distal end 170, and receiver 172; receiver 172 can be of any design useful to engage a dart and can either attach to or be integral with shaft 168.

Exemplary insertion tools for treatment of incontinence and vaginal prolapse are described, e.g., in U.S. patent application Ser. Nos. 10/834,943, 10/306,179; 11/347,553; 11/398,368; 10/840,646; PCT application number 2006/028828; and PCT application number 2006/0260618; each of which is incorporated herein by reference. Tools described in the cited patent documents are designed for placement of an implant in a pelvic region for the treatment of prolapse, male or female incontinence, etc., and may be modified to manipulate and a dart, at soft support tissue, pass the dart into or through the soft support tissue, and bring the dart into engagement with a receiver located at a distal end of a receiver tool, for subsequent manipulation of the dart by the receiver tool.

A different general type of useful insertion tool can combine the structures and functions of dart insertion tool and a receiver tool. An example can be a forceps or a pliers that includes two opposing jaws; one jaw is a dart insertion tool that can manipulate the dart and one jaw is a receiver tool that can manipulate (or integrally include) the receiver. FIG. 8 illustrates an insertion tool (i.e., forceps) for placing a dart. Forceps 200 includes two jaws, 206 and 208. Jaw 206 includes dart insertion tool, distal end 212, to engage a dart. Opposing jaw 206, the receiver tool, includes a receiver either removably attached to or integral to jaw 206. The jaws are located at a distal end of shaft 204 and one or both may pivot about point 207. At a proximal end of shaft 204 is handle 214, which can allow manipulation of (opening and closing of) jaws 206 and 208. In use, dart insertion tool (jaw 206) of forceps 200 can engage a dart attached to an extension portion of an implant. The distal end of forceps 200, including opposing jaws 206 and 208, can be passed through an incision, e.g., a vaginal incision, a perineal incision, a laporoscoic incision, an abdominal incision, or the like. The distal end can be placed at a region of soft support tissue. The jaws can be closed with soft support tissue located between dart (not shown) engaged at distal end 212 of jaw 206, and receiver 210. The dart can pass into the soft support tissue at an insertion location and tunnel through the soft support tissue. The receiver may optionally pass into the soft support tissue at an exit location, or may contact and support a surface of the soft support tissue. Both of the opposing jaws will contact the soft support tissue on the same side of the tissue. The dart contacts receiver 210, either within the soft support tissue or after the dart passes through the soft support tissue, and the dart becomes engaged, preferably securely engaged, with receiver 210. Jaw 206 is withdrawn from the dart and the dart can be manipulated by receiver 210.

A dart attached to an extension portion can be placed at and passed through soft support tissue of the pelvic region, to lead and pass the extension portion through the soft support tissue. The soft support tissue can be any tissue desired or useful to which to attach an extension portion, for example any of the following: muscle tissue of an obturator foramen (e.g., obturator internus muscle), tissue of an arcus tendineus or surrounding an arcus tendineus, tissue of a sacrospinous ligament, tissue in a region of a sacrospinous ligament, tissue of a coccyx region, tissue of a region of ischial spine, tissue of coccygeous muscle, tissue of iliococcygeous muscle, tissue of pubycoccygeus muscle, tissue of a uterosacral ligament, tissue of levator muscle, or combinations of these. Tissue in a “region” of an ischial spine can be tissue that is within one centimeter of an ischial spine, including tissue of the levator ani muscle (e.g., iliococcygeous muscle) and arcus tendineus.

When placing an extension portion through soft support tissue, embodiments of methods can lead the extension portion into a surface of soft support tissue at an insertion location, pass the extension portion through a mass of one or more types of soft support tissue, then cause the extension portion to exit the soft support tissue at an exit location at a nearby surface of soft support tissue. The insertion location and the exit location can both be located at surfaces at one side of the soft support tissue, generally at surfaces on the side of the tissue that can be accessed within the pelvic region, e.g., from a perineal incision, a vaginal incision, or an abdominal incision, or the like; this means that the extension portion enters on one side of tissue (in a first direction) (generally on the side accessible from a location within the pelvic region), passes laterally or “tunnels” sideways through a length of soft support tissue, then exits in a direction substantially opposite of the direction of insertion, returning into the pelvic region. The extension portion does not traverse soft support tissue by entering into one side of tissue, traversing the thickness of the tissue, and exiting the other side.

According to certain embodiments, the insertion and exit locations, at surfaces on the same side of soft support tissue, can be at surfaces of the same tissue, e.g., if both of the insertion and exit locations are located at surfaces of the same muscle, ligament, or tendon. For example, the extension portion enters soft support tissue at a surface on one side of coccygeus muscle; the extension portion passes laterally through a length of coccygeus muscle, e.g., tunneling sideways or laterally through the muscle; and the extension portion then exits the coccygeus muscle through an exit location at a surface on the same side of the muscle as the insertion location. Alternately, the extension portion can enter soft support tissue at a surface on one side of an obturator internus muscle; the extension portion can pass laterally through obturator internus muscle, e.g., tunneling sideways or laterally through the muscle; and the extension portion can then exit the obturator internus muscle through an exit location at a surface on the same side of the obturator internus muscle as the insertion location.

According to other embodiments of the invention, the exit location and the insertion location can be located on nearby, adjacent, or proximate locations of nearby or neighboring tissues, e.g., adjacent surface of different muscle, ligament, tendon, or combinations of these. For example, the extension portion can enter soft support tissue at a surface on one side of coccygeus muscle; the extension portion can pass through coccygeus muscle, e.g., tunneling sideways or laterally through the muscle and to a location behind a sacrospinous ligament; the extension portion can then exit from a surface of the sacrospinous ligament through an exit location on the side of the sacorospinous ligament that is adjacent to the insertion location on the coccygeus muscle.

Regardless of whether the extension portion passes through two different types of tissue or just a single tissue, embodiments of the invention can place an exit location near an insertion location, e.g., when tissue is unstressed, exit and insertion locations can be less than 2.5 centimeters apart, e.g., from 0.5 to 2.0 centimeters apart, or from 0.5 to 1.5 centimeters apart.

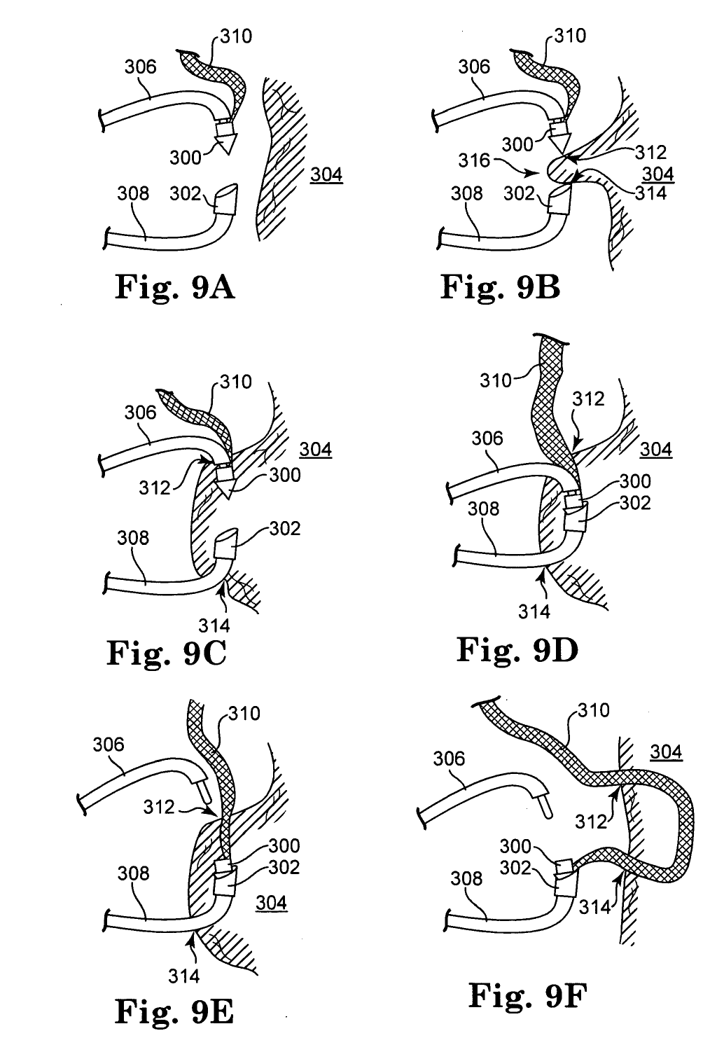

FIGS. 9A through 9F illustrate a general method of placing an exemplary dart by use of a method and receiver that involve the receiver penetrating into soft support tissue. FIG. 9A shows dart 300 and receiver 302, relative to tissue 304. Dart 300 may be as described herein, e.g. as illustrated at FIG. 1, FIG. 2A, or as otherwise described. Receiver 302 can also be as illustrated (e.g., at FIG. 3A or 5A) or as otherwise described, but as shown will include surfaces useful to penetrate and deflect tissue, and can be engaged with dart 300. Dart 300 is engaged at a distal end of dart insertion tool 306, and is also attached at a distal end of extension portion 310. Receiver 302 is attached to or integral with a distal end of receiver tool 308. Dart insertion tool 306 can be a jaw of a forceps, with an opposing jaw being receiver tool 308; alternately dart insertion tool 306 can be a separate tool relative to receiver tool 308.

FIG. 9B shows the use of tools 306 and 308 to place dart 300 and receiver 302 proximal to tissue 304. Dart 300 places pressure at a surface of tissue 304 at insertion location 312, while receiver 302 places pressure at a surface of tissue 304 at exit location 314. The opposing pressure of the dart and receiver grasping tissue 304 at the surfaces of the insertion and exit locations produces bulge 316 of soft support tissue. Insertion location 312 is preferably within 2.5 centimeters of exit location 314 when the tissue is unstressed and lies flat, e.g., as shown at FIGS. 9A and 9F.

FIG. 9C shows dart 300 and receiver 302 after each has penetrated into soft support tissue 304. FIG. 9D shows dart 300 and receiver 302 contacting one another within tissue 304 to become engaged. FIG. 9E shows dart insertion tool 306 withdrawn from dart 300 and, further, withdrawn from insertion location 312. FIG. 9F shows receiver tool 308 after being withdrawn from exit location 312. Extension portion 310 is placed within soft support tissue 304 by entry at insertion location 312, a tunneling or lateral tissue path extending within soft support tissue 304 a short distance (e.g., less than 3 centimeters), then exiting soft support tissue 304 through exit location 314.

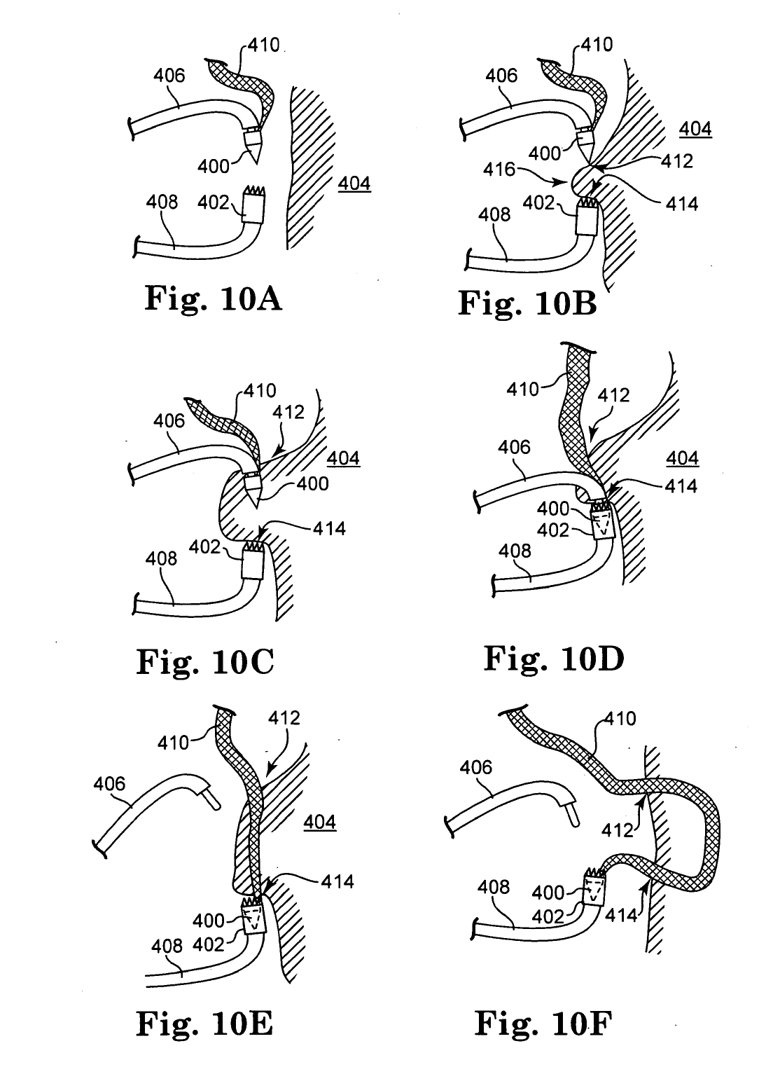

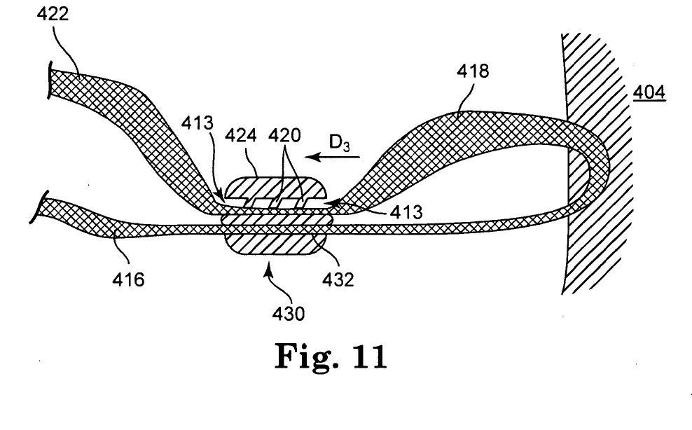

FIGS. 10A through 10F illustrate a general method of placing an exemplary dart by use of a method and receiver that involve a receiver that does not penetrate soft support tissue but that engages soft support tissue at a surface of an exit location. FIG. 10A shows dart 400 and receiver 402, relative to tissue 404. Dart 400 may be as described herein, e.g. as illustrated at FIG. 1, FIG. 2A, or as otherwise described. Receiver 402 can also be as illustrated (e.g., at FIG. 6A or 6C) or otherwise described, but as shown will include a distal surface that is frictionally capable of engaging a surface of soft tissue, and that can also become engaged with dart 400. Dart 400 is engaged at a distal end of dart insertion tool 406, and is also attached at an end of extension portion 410. Receiver 402 is attached to or integral with a distal end of receiver tool 408. Dart insertion tool 406 can be a jaw of a forceps, with an opposing jaw being receiver tool 408; alternately dart insertion tool 406 can be a separate tool relative to receiver tool 408.

FIG. 10B shows the use of tools 406 and 408 to place dart 400 and receiver 402 proximal to tissue 404. Dart 400 places pressure at a surface of tissue 404 at insertion location 412, and receiver 402 places pressure at a surface of tissue 404 at exit location 414, to produce bulge 416 of soft support tissue. The opposing pressure of the dart and receiver grasping tissue 404 at the surfaces of the insertion and exit locations produces bulge 416 of soft support tissue. Insertion location 412 is preferably within 2.5 centimeters of exit location 414 when the tissue is unstressed and lies flat, e.g., as shown at FIGS. 10A and 10F.

FIG. 10C shows dart 400 and receiver 402 after dart 400 has penetrated into soft support tissue 404; receiver 402 does not penetrate soft support tissue but exerts pressure onto a surface of soft support tissue 404 at exit location 414 to produce bulge 416, and maintains a position at that surface of soft support tissue 404, which is also the location at which dart 400 will exit soft support tissue 404, i.e., at exit location 414. FIG. 10D shows dart 400 after passing through soft support tissue 404 and exiting a surface of soft support tissue 404 at exit location 414; upon such exit, dart 400 becomes engaged with receiver 402. FIG. 10E shows dart insertion tool 406 withdrawn from dart 400 and, further, withdrawn from insertion location 412. FIG. 10F shows receiver tool 408 after being drawn away from the surface of soft support tissue 404 and exit location 412. Extension portion 410 is placed within soft support tissue 404 by entry at insertion location 412, a tunneling or lateral tissue path extending within soft support tissue 404 a short distance (e.g., less than 3 centimeters), then exiting soft support tissue 404 through exit location 414.

Optionally, according to a method as described herein, e.g., as illustrated at FIGS. 9A through 9F or 10A through 10F, a length of an extension portion between an insertion location of a soft support tissue and a tissue support portion can be adjusted, to adjust the position of or the tension place on a tissue support portion. Adjustment may be performed by passing a desired length of extension portion through the tissue path formed in the soft support tissue.

Also optionally a dart (e.g., 300 or 400) can be removed by cutting extension portion near the exit location. After adjustment a suture or other securing mechanism can optionally be placed through the extension portion at or near the exit location or the insertion location, to secure the extension portion to the soft support tissue.

Optionally, as illustrated at FIG. 11, an extension portion can include a clip that allows adjustment of the length of an extension portion, then secures the extension portion in place. The clip can be molded onto an extension portion along its length, and can have a channel through which a loose end of extension portion (the end passed through the soft support tissue) can be adjustably threaded. The loose end can be secured by the channel to prevent movement, either by a mechanical securing mechanism, or a one-way frictional mechanism such as teeth that allow movement of the loose end in one direction but not in the opposite direction.

Referring to FIG. 11, a cut-away side view of clip 430, clip 430 includes a bottom portion 432 molded or otherwise secured to extension portion 416. Extension portion 416 extends between tissue 404 and a tissue support portion (not shown). Clip 430 also includes top portion 242 and channel 413. Loose end 418, after being passed through soft support tissue 404, can be threaded through channel 413 and is allowed to pass in one direction (D3) while not being allowed to move in the opposite direction through the channel; movement in the direction opposite of D3 is prevented by teeth 420. End 422 is the end of the extension portion that included a dart; the extension portion at that end, near the dart, has been severed to remove the dart.

Embodiments of methods can be performed using any useful incision, e.g., a medial incision such as through a vaginal incision (for female anatomy) or perineal incision (for male anatomy), an abdominal incision, a laparoscopic incision, or another incision, and by use of a single insertion tool or a pair of insertion tools to placed the dart and extension portion.

An exemplary method for treating vaginal prolapse, e.g., anterior or posterior vaginal prolapse (including vaginal vault prolapse), can include transvaginally placing a tissue support portion at tissue of the anterior or posterior vagina or vaginal vault, and placing an extension portion extending from the tissue support portion to region of an ischial spine, preferably on both sides of a patient, and passing the extension portion through the soft support tissue. Alternate locations of placing extension portions can be at the sacrospinous ligament, levator ani muscle, or obturator foramen, with placement of the extension portion through the soft support tissue as described herein.

An exemplary method for treating male or female urinary (or fecal) incontinence can include transvaginally (female) or transperineally (male) placing a tissue support portion at tissue of the urethra (or rectum) and placing two opposing extension portions extending from the tissue support portion to opposing obturator foramen.

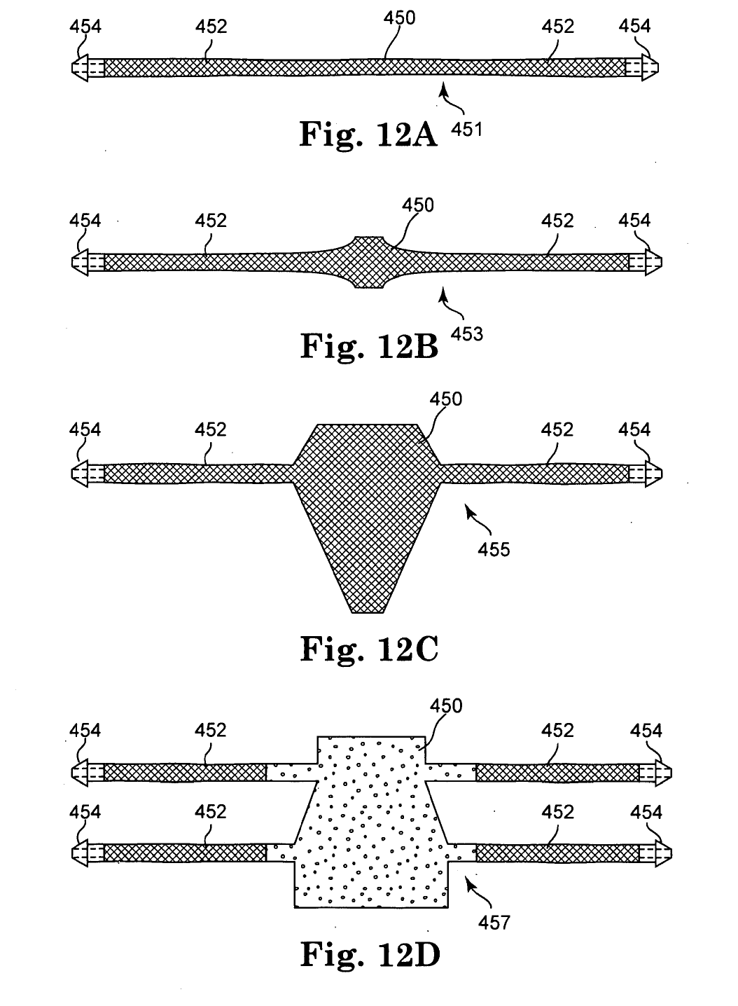

FIGS. 12A, 12B, 12C, and 12D illustrate embodiments of useful implants that include darts at distal ends of extension portions. Each implant includes a tissue support portion 450 (mesh or biologic), two or four extension portions 452 (of mesh, as illustrated), and darts 454.

Implants 451 and 453 of FIGS. 12A and 12B can be designed for treating male or female urinary or fecal incontinence. These exemplary implants can include supportive portions consisting of the tissue support portion and two opposing extension portions. Each is illustrated to include supportive portions that consist of a tissue support portion and either two extension portions.

Implant 455 at FIG. 12C can be designed for treating female vaginal prolapse, especially posterior vaginal prolapse. The exemplary implant includes supportive portions consisting of the tissue support portion 450 and two opposing extension portions 452.

Implant 457 at FIG. 12D can be designed for treating female vaginal prolapse, especially anterior vaginal prolapse. The exemplary implant can include supportive portions consisting of the tissue support portion 450 and four opposing extension portions 452. Alternate embodiments can include six extension portions. As illustrated, the tissue support portion is a biologic material and the extension portions are mesh.

Any of the method described can optionally and preferably be performed transvaginally, transperineally, laparoscopically, or trans-abdominally.

1. A pelvic implant system comprising a pelvic implant, a dart insertion tool, and a receiver, the implant comprising a tissue support portion, an extension portion, and a dart connected to the extension portion, the dart being capable of engaging the dart insertion tool, and the dart being capable of engaging the receiver.

2. The system of claim 1 wherein the dart comprises a proximal dart portion capable of engaging a distal end of the dart insertion tool, and a distal dart portion capable of engaging the receiver.

3. A system of claim 2 wherein the distal dart portion comprises a sharpened surface.

4. A system according to claim 2 wherein the distal dart portion comprises a deflecting surface.

5. A system according to claim 1 wherein
the dart comprises a surface that engages the receiver to produce a secure engagement between the dart and the receiver, and
the receiver comprises a surface that engages the dart to produce a secure engagement between the dart and the receiver.

6. A system according to claim 1 wherein the receiver comprises a distal receiver portion comprising a sharpened surface.

7. A system according to claim 1 wherein the receiver comprises a distal receiver portion comprising a deflecting surface.

8. A system according to claim 1 wherein the extension portion comprises mesh.

9. A system according to claim 1 wherein the receiver comprises a distal receiver portion comprising a surface capable of frictionally engaging a surface of soft support tissue without penetrating the soft support tissue.

10. A system according to claim 1 wherein the dart insertion tool comprises a handle, a shaft, and a distal end that engages the dart in a manner that allows the dart insertion tool to push the dart.

11. A system according to claim 1 wherein the receiver tool is separate from the dart insertion tool, and the receiver tool comprises a handle, a shaft, and a receiver integral with the shaft.

12. A system according to claim 1 comprising a forceps comprising two opposing jaws, one jaw comprising the dart insertion tool and the opposing jaw comprising the receiver tool.

13. A system according to claim 1 wherein the implant comprises supportive portions consisting of a tissue support portion and four extension portions, each extension portion comprising a dart.

14. A system according to claim 1 wherein the implant comprises supportive portions consisting of a tissue support portion and two extension portions, each extension portion comprising a dart.

15. A pelvic implant system comprising a pelvic implant, a dart insertion tool, and a receiver, the pelvic implant comprising a tissue support portion, an extension portion, and a dart connected to the extension portion, the dart comprises insertion tool engaging means capable of engaging a distal end of the dart insertion tool, and receiver engaging means capable of engaging the receiver.

16. A method for treating a pelvic condition, the method comprising
providing an implant comprising a tissue support portion, an extension portion, and a dart attached to the extension portion,
placing the tissue support portion in contact with pelvic tissue,
extending the extension portion to soft support tissue,
inserting the dart into the soft support tissue at a surface of the soft support tissue,
passing the dart through soft support tissue, and
causing the dart to exit the soft support tissue from a surface on the same side of the soft support tissue into which the dart was inserted.

17. A method according to claim 16 comprising
pushing the dart into soft support tissue at an insertion location on a surface of the soft support tissue,
pushing a receiver into the soft support tissue at an exit location at a surface of the soft support tissue on the same side of the soft support tissue as the insertion location,
engaging the dart and the receiver at a location within the soft support tissue, and
using the receiver to pull the dart through the soft support tissue and to cause the dart to exit the soft support tissue at the exit location.

18. A method according to claim 16 comprising
pushing the dart into soft support tissue at an insertion location on a surface of the soft support tissue,
placing a receiver at a surface of the soft support tissue, the surface being on the same side of the soft support tissue as the insertion location,
passing the dart through soft support tissue,
causing the dart to exit the soft support tissue at an exit location, to cause the dart to engage the receiver at a location external to the soft support tissue.

19. A method for treating a pelvic condition, the method comprising
providing an implant comprising a tissue support portion and an extension portion,
placing the tissue support portion in contact with pelvic tissue,
extending the extension portion to soft support tissue,
inserting the extension portion into the soft support tissue at an insertion location,
passing the extension portion through the soft support tissue, and
causing the extension portion to exit the soft support tissue at an exit location on the same side of the soft support tissue as the insertion location,
wherein the distance between the insertion location and the exit location is not greater than 2.5 centimeters.

20. A method of surgically implanting a pelvic implant, the method comprising
providing a system according to claim 1,
placing the tissue support portion in contact with a pelvic tissue,
extending the extension portion to soft support tissue,
inserting the dart into the soft support tissue by penetrating a surface of the soft support tissue at an insertion location,
passing the dart through soft support tissue to an exit location on a surface of the soft support tissue that is on the same side of the soft support tissue as the insertion location, and
engaging the dart and the receiver.

21. A method of surgically implanting a pelvic implant, the method comprising
providing a system according to claim 12, wherein the dart insertion tool and the receiver are opposing jaws of the forceps,
engaging the dart with a jaw of the tool, and contacting the engaged dart with soft support tissue at an insertion location,
contacting a receiver with soft support tissue at an exit location,
closing the jaws of the forceps to grasp soft support tissue between the two opposing jaws to produce a bulge of soft support tissue between the insertion location and the exit location,
passing the dart through the soft support tissue, and
engaging the dart and the receiver.

22-31. (canceled)
